(19) United States
(12) Patent Application Publication
(10) Pub. No.: US 2011/0144765 A1
(43) Pub. Date: Jun. 16, 2011

(54) Process For Producing Porous Scaffolds From Sinterable Glass

(75) Inventors: Julian Jones, London (GB); Robert Graham Hill, Maidenhead (GB); Zoe Yunxie Wu, London (GB)

(73) Assignee: IMPERIAL INNOVATIONS LIMITED, London (GB)

(21) Appl. No.: 12/994,483

(22) PCT Filed: May 27, 2009
(86) PCT No.: PCT/GB09/01325

§ 371 Date: Feb. 23, 2011

(30) Foreign Application Priority Data
May 27, 2008 (GB) 0809577.0
May 27, 2008 (GB) 0809578.8

(51) Int. Cl. A61F 2/28; C03C 3/00; C03B 19/06; C03B 19/08

(52) U.S. Cl. 623/23.61; 501/11; 65/22

(57) ABSTRACT

The invention relates to a process for producing a porous glass construct with interconnected porosity, the resulting porous construct and its use as a macroporous scaffold in bone repair and regeneration.

The present invention relates to a process for producing a porous glass construct with interconnected porosity and strength suitable for bone ingrowth and bone regeneration, the resulting porous construct and its use as a macroporous scaffold in bone repair.

As healthcare is improving and life expectancy increases we are outliving our body parts, including our bones. Bone grafting procedures are used to regenerate bone that has been removed or damaged due to disease and trauma. More than 300 000 bone graft operations are performed in Europe each year. Current surgical best practice is to remove healthy bone from the iliac crest (autograft), and place it into the desired location. While effective, this procedure requires additional surgical time (an extra invasive operation) and can produce post-operative pain at the site of bone removal and a long recovery time. The bone is also in limited supply. A more plentiful supply of bone are allografts; bone sourced from bone banks, which distribute bone from cadavers. These bones do not usually have the mechanical strength of autografts and there is a chance of immunorejection and disease transmission. A patient may require lifetime treatment with expensive immunosuppressant drugs that can also yield dangerous side effects. Animal bones (xenograft) can also be used, e.g. freeze dried bovine bone, but mechanical properties are poor and there is still the risk of disease transmission.

Bone grafts are used in: (i) maxillofacial surgery, (ii) in orthopaedics to repair defects created due to trauma, tumours and cysts, and (iii) in dentistry, where they are often used to cure periodontitis (bone loss at the tooth root). Many surgical procedures of the spine, pelvis and extremities require grafts. Bone grafts may also be needed in situations where healing may be difficult due to nicotine use, or the presence of diseases such as diabetes or autoimmune deficiencies.

A regenerative scaffold is particularly important in the elderly and in the young. All tissues in elderly people are slow to heal due to lack of active cells. Therefore a synthetic bone-healing material that is available off the shelf for a surgeon to immediately implant into a bone defect would dramatically improve quality of life of patients across the globe.

One of the most common uses of bone grafts in spine surgery is during spinal fusion, which is a vital operation needed to reduce debilitating pain. One of every 700 newborns has a cleft pallet. Maxillofacial surgery with materials that respond to the physiological environment are vital so that the regenerative site can remodel as the child grows.

Biomaterials can be used in biomedical applications, specifically tissue regeneration and tissue engineering, and can replace bone grafts. Such regenerative bone graft substitutes have the potential to greatly improve healthcare treatments and quality of life of patients. A biologically active (or bioactive) material is one which, when implanted into living tissue, induces formation of an interfacial bond between the material and the surrounding tissue.

Typically strategies for promoting bone regeneration involve use of a scaffold material. A scaffold is a template on which bone can grow in three dimensions (3D), creating a construct of tissue and scaffold. The two main bone regeneration strategies involving use of a scaffold are in situ tissue regeneration and tissue engineering. Commonly, tissue engineering involves growing cells on a scaffold in a bioreactor outside the body and then implanting the scaffold, after which the scaffold should dissolve as the bone remodels into mature bone. In in situ tissue regeneration, a scaffold is implanted directly into the body. In both cases, the implanted scaffold materials must adapt to the physiological environment. An ideal scaffold for bone repair should: 1) act as template for bone growth in three dimensions; 2) be biocompatible (not toxic); 3) form bonds with host bone (a property referred to as “bioactivity”) and stimulate bone growth; 4) dissolve at a controlled rate with non-toxic degradation products; 5) have mechanical properties matching that of the host bone on implantation; and 6) be capable of commercial production and sterilisation for clinical use.

In order to fulfil criterion 1, the scaffold should have a pore network that is interconnected in 3D, with interconnections large enough to allow cell migration, fluid flow (nutrient delivery), and bone to grow in 3D. The minimum interconnect size for bone with a blood supply to grow in is thought to be 100 μm.

Cells require signals to stimulate them to lay down new tissue. The signals are usually provided by growth factors or hormones. In bone tissue engineering, the signal can either be provided by additives to the bioreactor or delivered by the material. For in situ bone regeneration, they must be delivered by the material.

A material that has the potential to fulfil many of the criteria for an ideal scaffold for use in hard tissue repair is bioactive glass. The first bioactive glass was discovered by Hench and was termed Bioglass®. It obtained FDA approval in 1993 and has been used clinically as a regenerative bone filling powder under the product names Perioglas® and Novabone®. Bioactive glasses bond to bone because a hydroxycarbonated apatite (HCA) layer forms on their surface on contact with body fluid. HCA is similar in composition to bone mineral and forms a strong bond therewith. Bioactive glasses dissolve safety in the body, releasing critical concentrations of ions which act to signal cells even when few active cells are present. This is particularly important for older patients. By means of example, silicon and calcium ions have been found to signal osteogenic cells to produce new bone, strontium is also known to stimulate bone regeneration and zinc is an antibacterial agent.

Bone growth and blood vessel development can be triggered by biological growth factors, but these are difficult to deliver in vivo. Sustained delivery is especially difficult. Bioactive glasses therefore have advantages over materials that deliver biological growth factors: resorbable glasses can deliver active ions at controlled rates; they are cheaper to produce than growth factors; they will have a longer shelf life and are easier to store and transport than biologicals are.

Whilst bioactive glasses are suitable for use as regenerative materials, the Bioglass® composition is unsuitable for the production of porous scaffolds. This is because a sintering process must be employed, which requires glasses to be heated above their glass transition temperature in order to initiate localised flow. The Bioglass® composition crystallises immediately above its glass transition temperature and once Bioglass® crystallises, its bioactivity and degradation rate become unpredictable.

There are two types of bioactive glass; melt-derived and sol-gel derived. By foaming sol-gel derived silica based bioactive glasses, porous scaffolds have been developed (WO02/096391). These scaffolds fulfil many of the required criteria. They have interconnected pore networks that are similar to trabecular bone (Jones et al., Biomaterials 28: 1404-1413, 2007) that is ideal for bone regeneration. Cell response studies on such scaffolds have found that primary human osteoblasts lay down mineralized immature bone tissue thereon, without additional signalling species. Bioactive glasses provide signals, in the form of release of silicon and calcium ions, required for these processes to occur.

Sol-gel derived bioactive glass scaffolds can largely fulfil the criteria for an ideal scaffold, apart from their mechanical properties. They also degrade more rapidly than melt-derived glasses. In certain applications, however, a slow degradation rate is necessary, especially when mechanical support is required long term. Previous attempts to produce porous melt-derived bioactive glass scaffolds have had limited success. Livingston et al. (Livingston et al., J. Biomed. Mat. Res. 62(1): 1-13, 2002) mixed 45S5 melt-derived bioactive glass (Bioglass®) powders, with particle size range of 38-75 μm, with 20.2 wt % camphor (C₁₀H₁₆O) particles, with particle size range of 210-350 μm. The mixture was dry-pressed at 350 MPa and heat treated at 640° C. for 30 minutes. The camphor decomposed to leave porous Bioglass® blocks. However total porosity was just 21%, which is not high enough to create an interconnected pore network. It is not clear whether this material was actually amorphous. Porous scaffolds with interconnected pore morphologies have been made by the polyurethane foam replication process, where a polymer foam was coated with Bioglass® powders and heat treated to remove the polymer and sinter the glass. However, on sintering, the glass crystallised to form a glass-ceramic (Chen et al, Biomaterials 27(11): 2414-2425, 2006). Fu et al. ((Fu et al., J. Biomed. Mat. Res 82A(1): 222-229, 2007) slip cast particles (255-325 μm) of the 13-93 glass composition (53 wt % SiO₂, 6 wt % Na₂O, 12 wt % K₂O; 5 wt % MgO, 20 wt % CaO, and 4 wt % P₂O₅). The particles were dispersed in a polyvinyl alcohol (PVA) solution and cast into moulds. The heating regime removed the PVA binder and sintered the particles to form porous blocks. Although XRD showed no crystallisation of the glass, the percentage porosity was too low (40-45%) to obtain high interconnectivity. The pore network did not resemble that of trabecular bone.

Bioactive glass compositions that can be sintered have recently been developed (WO2007/144662). These compositions extend the temperature window between the glass transition temperature and the crystallisation temperature, allowing sintering to be performed. These glasses were used to form porous scaffolds in a process which involves the used of polymethylmethacrylate (PMMA) spheres as a space holder. PMMA spheres were mixed with bioactive glass powders and cold pressed into a pellet. The pellet was then calcined by heating to 700° C. to burn out the polymer and sinter the glass. The glass remained amorphous but the pores were of low connectivity. Moreover, use of the foam replication technique with the sinterable glasses described in WO2007/144662 produces a material with hollow struts (foam walls), reducing the mechanical strength of the material detrimentally to its use as a scaffold.

The above examples demonstrate that there are complex problems to solve in the development of porous melt-derived bioactive glass scaffolds. There is a need for a biocompatible porous scaffold that can act as template for bone growth in three dimensions, that has the appropriate mechanical properties to allow use for bone regeneration in load bearing sites, that is degradable at a controlled rate, that contains a source of calcium ions to provide bioactivity and stimulate bone growth and that is capable of commercial production and sterilisation for clinical use. It has now been determined that a porous material fulfilling these criteria for use as a scaffold can be produced by using a gel cast foaming technique, which involves providing a slurry containing particulate glass, foaming the glass particulate slurry with a surfactant and gelling the foam by in situ polymerisation of gelling agents, to give glass particles within a polymer matrix. The foam can be poured into a mould immediately prior to gelation and heat treated to remove the polymer and sinter the glass particles, creating a solid glass foam with dense struts.

Accordingly, in a first aspect the present invention provides a process for the production of a porous material, the process comprising:
a) forming a slurry comprising melt-derived glass particles, a monomer, a cross-linker and an initiator in a solvent;
b) adding a surfactant and a catalyst to the slurry;
c) agitating the slurry in the presence of a gas (for example air) to generate a foam;
d) drying the foam; and
e) sintering the dried foam to provide a porous glass scaffold.

In a preferred embodiment, the melt-derived glass is biologically compatible. Preferably the glass is a bioactive glass. In the context of this invention, the glass used is preferably a sinterable glass. A glass is sinterable if it can be sintered (i.e. heated to the sintering temperature) without significant crystallisation, and preferably with no crystallisation. The sintering temperature will therefore be a temperature above the glass transition temperature (Tg) but below the onset temperature for crystallisation (Tconset). These values can be determined experimentally for a glass using high temperature differential scanning calorimetry. In order for a glass to be sinterable it should preferably have a processing window between the glass transition temperature (Tg) and the crystallisation onset temperature (Tconset) of at least 50° C., more preferably at least 100° C. and even more preferably at least 150°.

Accordingly, the porous material produced by the process of the invention comprises a glass which has maintained its amorphous glass structure.

Some crystallisation can be tolerated for certain applications, but preferably the sintered glass in the porous scaffold is at least 90% amorphous, preferably 100% amorphous. The percentage of amorphous glass present in the scaffold can be determined by integration of the area of the diffraction peaks over the total integrated area of amorphous scattering seen by XRD analysis of the scaffold.

In the slurry, the monomer reacts with the crosslinker, beginning the formation of a polymer network. This polymerisation causes the viscosity of the slurry to increase and as the viscosity increases the glass particles begin to bind together. The surfactant is added while the viscosity increases and after addition of the surfactant the catalyst is added. The resulting slurry is then foamed by agitating it vigorously. The surfactant acts to reduce the surface tension of the solution thereby stabilising air bubbles formed. Drying of the foam is followed by sintering, which acts to remove the polymerised material and sinter the glass particles together, leading to the production of a porous material formed from the glass. Drying of the foam before sintering is necessary to drive the solvent out from within the foam and stabilise the foam. Without the drying step, sintering would lead to rapid solvent evaporation which would damage the foam structure. The order of addition of the various components to the slurry is important. The glass particles, monomer, cross-linker and initiator should be added to the slurry before the surfactant. The catalyst, which acts to significantly increase the speed of polymerisation is the last component to be added. Advantageously, the process of the invention achieves a gelling rate that allows foaming to take place and a structure to be obtained that does not collapse or crystallise on sintering. Factors that influence the foaming process include slurry concentration, initiator concentration, viscosity of the solution and surfactant type and quantity.

In a preferred embodiment, the solvent is water. The solvent allows the surfactant to stabilise the foam prior to gelation. It is preferable for the slurry to be prepared immediately prior to processing (i.e. addition of surfactant, catalyst and foaming), such that the particulate glass is in contact with water for a maximum of 5 minutes prior to gelation.

The monomer may be present at 2.2-44.4% (w/v) and the cross-linker may be present at 1.1-22.2% (w/v), based on the total slurry volume. This equates to 1-20 g monomer and 0.5-10 g cross linker in 45 ml total slurry volume. Preferably, the monomer is present at 5-20% (w/v), preferably 10-15% (w/v), and the cross-linker is present at 3-10% (w/v), both based on the total slurry volume. Preferably, the ratio of the monomer to cross-linker is 2:1 (by wt).

In a preferred embodiment, the monomer is methyl methacrylate (MMA). In a preferred embodiment, the cross-linker is N,N′-methylenebisacrylamide.

In a preferred embodiment, the initiator is ammonium persulfate (APS). The more fresh the APS solution, the faster the gelling time. Preferably, the APS is provided as an aqueous solution, preferably at a concentration of 0.52 g/ml. When this concentration of APS solution is used, the loading of the APS solution within the total slurry volume may be from 1.1-22.2% (v/v), preferably from 1.1-11.1% (v/v), more preferably 1.1-8.9% (v/v), more preferably 3.3-5.6% (v/v). These values equate to the APS solution being provided at a volume of 0.5-10 ml, preferably at 0.5-5 ml, more preferably 0.5-4 ml, most preferably 1.5-2.5 ml, in a total slurry volume of 45 ml. The total slurry volume is the total volume of the glass particles, solvent, monomer, crosslinker, initiator, surfactant and catalyst, prior to foaming. It will be appreciated that a different concentration of APS solution could be used and in this case the volume of APS solution provided within the slurry would be calculated to provide the same total concentration of APS within the slurry.

In a preferred embodiment, the catalyst is N,N,N′,N′-tetramethylene diamine (TEMED).

In a preferred embodiment, the surfactant is Triton X-100. Use of a surfactant stabilises bubbles formed within the slurry as it is foamed by lowering surface tension. This means the bubbles formed are larger than they could be without the presence of surfactant. It is during the gelation process that the pores of the resulting porous material are set and the use of a surfactant allows for the production of a material with large and connected pore structure, a feature required for bone graft replacement materials and tissue engineering scaffolds, where large pores are needed to allow tissue ingrowth. In a total slurry volume of 45 ml, the surfactant may be present at 0.001-1 ml (i.e. a v/v % of 0.0022-2.2%). Preferably, the surfactant is present at 0.1-1 ml (0.22-2.2% v/v).

Both the water content of the slurry and the glass content are important in controlling the foam volume achieved during agitation and consequently the pore sizes in the resulting porous material. The foam volume is proportional to the resulting pore size. In a preferred embodiment, the content of glass particles in the total volume of the slurry is from 22% to 67% (w/v), preferably from 30% to 50% (w/v), for example from 30% to 40% (w/v) or 40% to 50% (w/v). In certain embodiments, a content of 42% to 46% (w/v) is utilised, preferably 44% (w/v).

In addition, the catalyst content of the slurry has a significant effect on the gelling time of the slurry, with gelling occurring faster as catalyst concentration is increased. This, in turn, has an effect on the foam volume, with increasing catalyst content leading to a decrease in foam volume. In a preferred embodiment, the catalyst is 6.63M TEMED, provided at a 4.4% to 13.3% v/v content with respect to the total slurry volume. It will be appreciated that if TEMED is provided at a different molarity, the volumes used can be adjusted to provide a corresponding catalyst concentration in the slurry.

The particle size of the glass particles has an important influence of the success of sintering. The maximum particle size (the maximum particle diameter) of the glass particles is determined by the sieving the glass particles through a sieve. In a preferred embodiment, the maximum particle size of the glass is no greater than 100 μm (for example achieved by passing the glass particles through a 100 μm sieve). Preferably, the maximum particle size is 38 μm.

It is preferable for the glass to be provided with a range of particles sizes such that the smaller particles will act to fill gaps between the larger particles. Controlling particle size ensures that the walls formed in the resulting porous material are not too thick to achieve the desired high porosity. Tg is independent of particle size whilst crystallisation occurs predominantly by a surface nucleation process. Consequently Tconset decreases with decreasing particle size. As particle size is reduced, surface area increases and the energy associated with this surface drives the sintering process. Neglecting crystallisation onset temperature, the smaller the particle size, the easier and lower the sintering temperature. The invention achieves a balance between the sintering temperature and crystallisation.

In a preferred embodiment, the porous material formed in step e) is treated with simulated body fluid. This will cause apatite to be formed on the material's surface and consequently in use of the porous material will minimise the pH rise on incorporation of cells and aid osteoblast attachment.

In a preferred embodiment, the glass is formed from SiO₂ (30-60 molar %), a source of calcium (0-50 molar %), a source of sodium (0-30 molar %), a source of potassium (0-30 molar %), a source of zinc (0-10 molar %), source of magnesium (0-20 molar %) and P₂O₅ (0-14 molar %).

Throughout this description of the invention, percentages of glass components are molar percentages. The sources of calcium, sodium, potassium, zinc and magnesium are each independently the respective oxide (CaO, Na₂O, K₂O, ZnO and MgO) or a compound that decomposes to form the oxide. Thus, where glass compositions are referred to throughout the application as comprising a certain molar percentage of an oxide, when forming the glass, the oxide can be provided as the oxide per se or as a compound that decomposes to the oxide. Accordingly, a glass having the composition defined above can be described as comprising 30-60 mol % SiO₂, 0-50 mol % CaO, 0-30 mol % NaO, 0-30 mol % K₂O, 0-10 mol % ZnO, 0-20 mol % MgO and 0-14 mol % P₂O₅.

Preferably, the glass comprises 46-50% SiO₂. Preferably, the combined molar percentage of Na₂O and K₂O is 5-15%. The glass may also comprise 20-50 mol % CaO, preferably 20-45 mol % CaO. Preferably, the combined molar percentage of ZnO, MgO, CoO, SrO and P₂O₅ within the glass is 1-12%. In certain embodiments, at least 0.5 mol % P₂O₅ is present.

In a preferred embodiment, the glass comprises from approximately 46 to 50% SiO₂, approximately 0.5% to 1.5% (preferably approximately 1%) P₂O₅, approximately 0 to 2% B₂O₃, approximately 8 to 40% CaO, approximately 0 to 15% SrO, approximately 5 to 7% Na₂O, approximately 4 to 7% K₂O, approximately 0 to 4% ZnO, approximately 0-4% MgO and approximately 0 to 9% CaF₂.

Preferably, the glass comprises 2-4% ZnO. Preferably, the glass comprises 2-4% MgO.

In an even more preferred embodiment, the glass comprises approximately 46 to 50% SiO₂, approximately 0.5% to 1.5% P₂O₅, a total molar percentage of CaO, ZnO, MgO and SrO of approximately 35-40% and approximately 5 to 7% Na₂O and approximately 5 to 7% K₂O.

In a preferred embodiment, the glass additionally comprises a source of cobalt ions, for example CoO, at a molar percentage up to 5%. Copper may be used as an alternative to cobalt.

In certain embodiments the glass comprises a source of strontium ions (for example SrO). The source of strontium ions can replace some or all of the source of calcium ions. Accordingly, the glass may comprise a combined CaO and SrO content of 0-50 mol %, preferably 20-50 mol %, more preferably 25-40 mol %. In some embodiments, up to 5 mol % of the total glass composition is SrO. Strontium ions are useful for promoting bone regeneration. In addition, the mixture of both strontium and calcium species restricts crystallisation of the glass slightly and aids processing.

Advantageously, the glass compositions used in the present invention allow the glass to sinter without crystallisation occurring. It is also desired for the glass to be bioactive. To achieve bioactivity, network connectivity of the glass should be close to 2.0, which effectively specifies a silica mole fraction preferably below 50 mole %. Sinterable glasses can be produced by going to higher SiO₂ contents, increasing the glass crosslinking and thus restricting its tendency to crystallise. However, this is at the expense of bioactivity. One way to combat this is to include ZnO and/or MgO in the glass composition. These go into the silicate network structure reducing bioactivity very slightly but dramatically retarding crystallisation and increasing Tconset. Preferably, K₂O and SrO are also included. The more components in the glass, the greater the entropy of mixing and the more the disordered glassy state is stabilised at the expense of the ordered crystalline state.

Thus, a suitable glass composition for producing a highly porous material that is also bioactive is a balancing act between many factors. A further factor is the total alkali metal content which, if too high, facilitates crystallisation. Moreover, too much ZnO and MgO will reduce bioactivity. In certain embodiment, bioactivity can be increased by substituting Sr for Ca and offsetting the loss seen by inclusion of Mg and Zn.

In certain embodiments, the step of drying the foam is carried out at a temperature from 50° C. to 200° C., preferably from 100° C. to 200° C., more preferably from 115° C. to 160° C. In some embodiments, the drying temperature is from 120° C. to 155° C. or from 120° C. to 130° C., for example 125° C.

In a preferred embodiment, the sintering process is a viscous flow sintering process.

In certain embodiments of the invention, the sintering temperature is from 400° C. to 900° C., preferably from 600° C. to 800° C., more preferably from 630° C. to 730° C. In some embodiments, the sintering temperature is 700-750° C., whereas in other embodiments, the sintering temperature is 680-700° C. Accordingly, in some embodiments, the sintering temperature is from 630° C. to 730° C. (preferably 680-700° C.) and the drying temperature is 120-130° C.

In certain embodiments, sintering is carried out in a two-step process comprising heating the foam to a first hold temperature at which polymer is removed from the foam, followed by increasing the temperature to a sintering temperature and maintaining the sintering temperature which causes sintering of the glass particles. The sintering temperature is as defined above and the first hold temperature may be from 80-800° C., preferably 100-400° C., more preferably 200-400° C. In certain embodiments, the first hold temperature is 150-200° C. In other embodiments, the first hold temperature is 300-400° C., preferably. 340-360° C., for example 350° C.

The various drying, hold and sintering temperatures described above can be used in any combination thereof. For example, the sintering temperature may be 600-800° C. (preferably, 630-730° C. or 680-700° C.), the drying temperature may be 120-130° C. and the first hold temperature may be 300-400° C.

The two-step sintering process may involve maintaining the foam at the first hold temperature for a first dwell time, which preferably is up to 24 hours, preferably 0.5 to 1.5 hours, and maintaining the foam at the sintering temperature for a sintering time, which is preferably up to 24 hours, preferably up to one hour, more preferably from 0.4 to 0.6 hours. The sintering conditions and the process of sintering differ from the process used for producing ceramic foams based on, for example, hydroxyapatite where the sintering process does not occur by viscous flow sintering and much higher temperatures of around 1100-1250° C. are typically required

Preferably, the temperature is increased to the first dwell temperature at a rate of 0.05-200° C./minute, preferably 0.05-5° C./minute. Preferably, the temperature is increased from the first hold temperature to the sintering temperature at a ramp rate of 0.05-200° C./minute, preferably 0.05-5° C./minute.

After sintering, the resulting porous material is cooled, preferably at maximum cooling rate of 60° C./min.

It should be appreciated that various factors within the sintering process can be varied. For example, a lower sintering temperature can be employed with a lower ramp rate and a longer sintering time.

Preferably, sintering is carried out in a furnace in an oxygen containing environment. This is in order to remove carbon residues. In a preferred embodiment, the porous material has an interconnected pore network making it suitable for use as a scaffold for promoting bone growth. Preferably, the porous material comprises macropores having a mean diameter up to 500 μm, preferably between 100 and 500 μm. Preferably, the mean minimum dimension of interconnection between macropores is at least 100 μm.

In a preferred embodiment, the glass additionally comprises a source of metal ions useful for promoting wound healing and/or revascularisation, for example lithium or copper ions.

In a second aspect, the present invention provides a porous material formed from a melt-derived glass, wherein the amorphous glass network is present within the porous material and wherein the porous material comprises macropores having a mean diameter up to 500 μm, preferably between 100 and 500 μm. Preferably, the mean minimum dimension of interconnection between macropores is at least 100 μm. Preferably, the porous material is as produced by the process of the first aspect of the invention.

In a third aspect, the present invention provides a porous material as produced by the process of the first aspect of the invention.

In a fourth aspect, the present invention provides a porous material of the second or third aspects of the invention for use in medicine. Preferably, the material is for use as a scaffold for aiding bone repair and/or regeneration.

In a fifth aspect, the present invention provides a bone graft substitute or a tissue engineering scaffold comprising a porous material of the second or third aspects of the invention.

All preferred features of each of the aspects of the invention apply to all other aspects mutatis mutandis.

The invention may be put into practice in various ways and specific embodiments will be described to illustrate the invention, with reference to the accompanying figures in which:

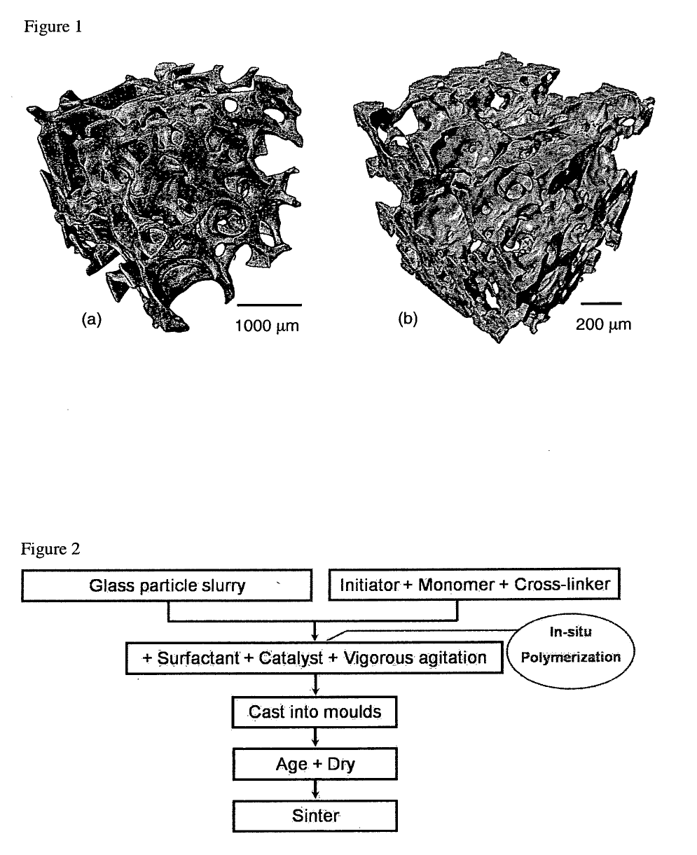

FIG. 1 shows three dimensional (3D) X-ray micro computer tomography (μCT) images of human trabecular bone (FIG. 1a) and a melt-derived bioactive glass scaffold produced by the gel casting foaming process of the invention (FIG. 1b) and shows that the pore network of the scaffolds are very highly interconnected and similar to the pore structure of trabecular bone. The macrostructure of the glass can be tailored depending on the relative amounts and the type of gelation catalyst, initiator (gelling agent) used in preparation of the material.

FIG. 2 shows a flow chart of the gel-cast foaming process of the invention.

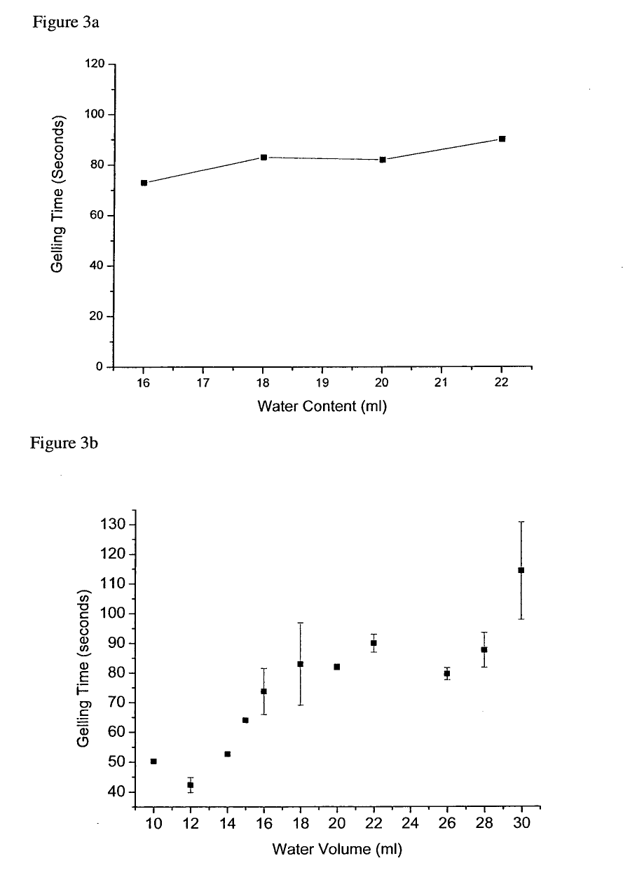

FIG. 3 shows a graph of gelling time as a function of water content in the slurry, FIG. 3b is an expanded versions of FIG. 3a.

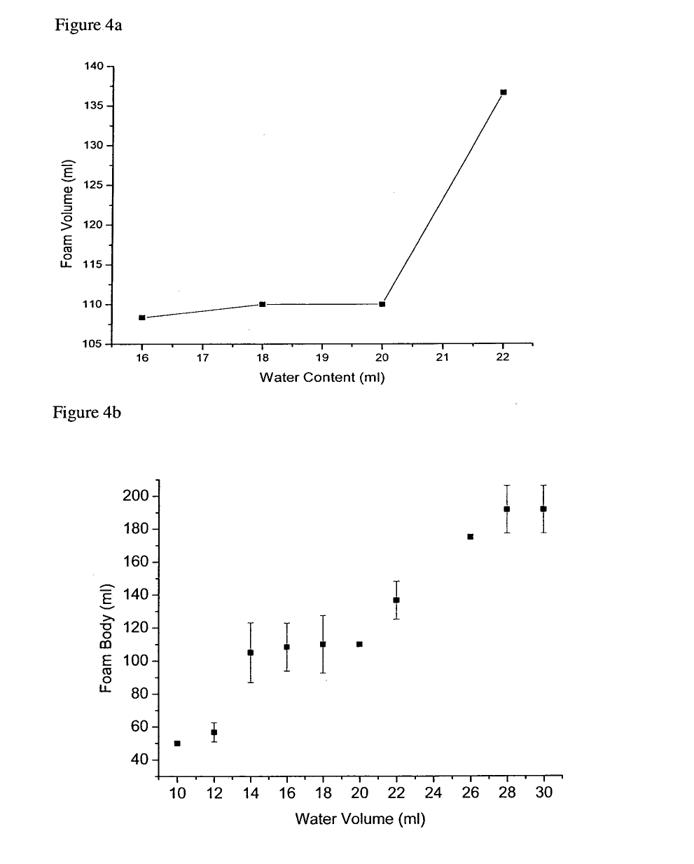

FIG. 4 shows a graph of foam volume achieved by foaming 45 ml of slurry as a function of water content, FIG. 4b is an expanded version of FIG. 4a.

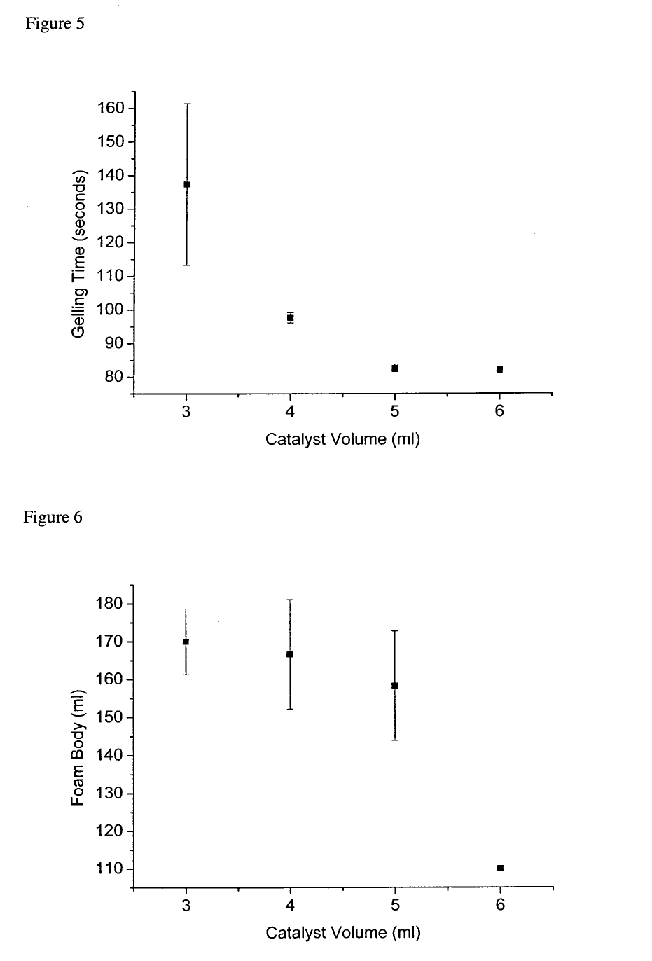

FIG. 5 shows a graph of gelling time as a function of catalyst content.

FIG. 6 shows a graph of foam volume achieved from 45 ml of slurry as a function of catalyst content.

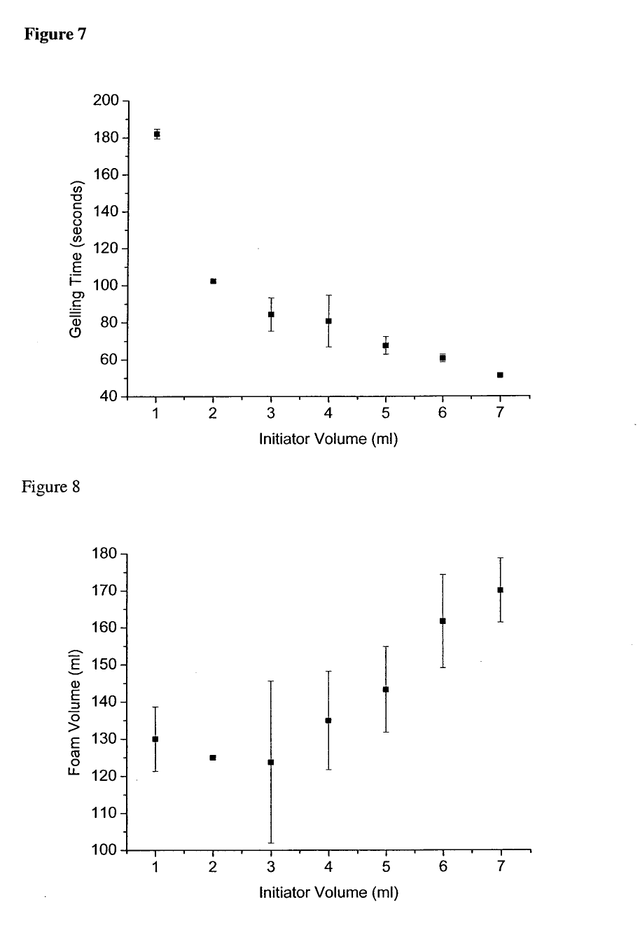

FIG. 7 shows a graph of gelling time as a function of initiator content.

FIG. 8 shows a graph of foam volume achieved from 45 ml of slurry as a function of initiator content.

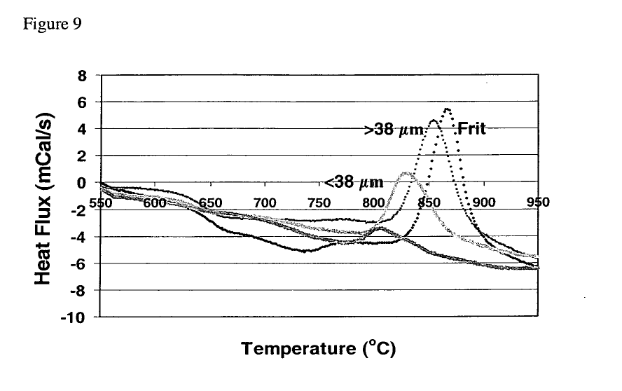

FIG. 9 shows DSC traces of ICIE16M at different particle sizes.

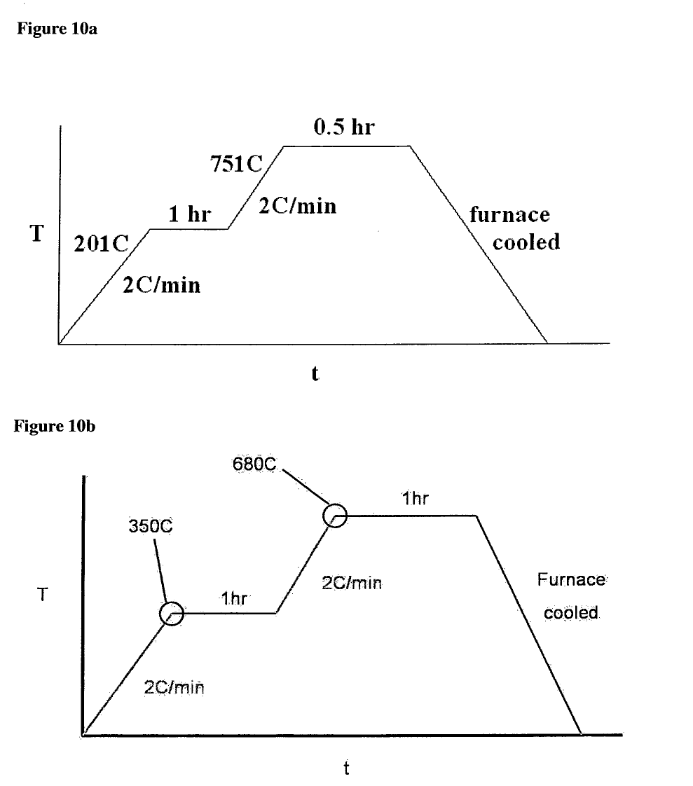

FIGS. 10a and 10b show schematics of successful sintering programmes for ICIE16M.

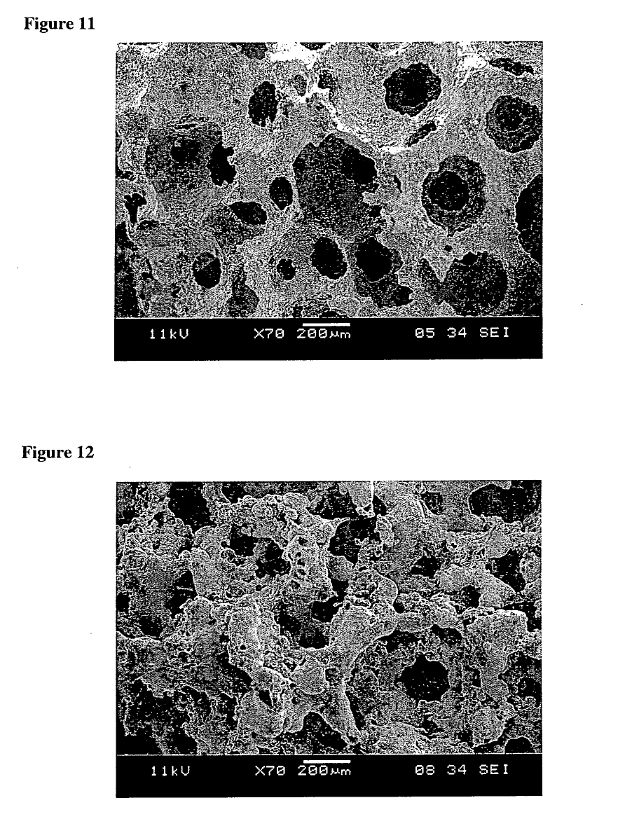

FIG. 11 shows a SEM image of an ICIE16M gel-cast foam scaffold before sintering.

FIG. 12 shows a SEM image of an ICIE16M gel-cast foam scaffold after sintering, produced using a particle size of >38 μm.

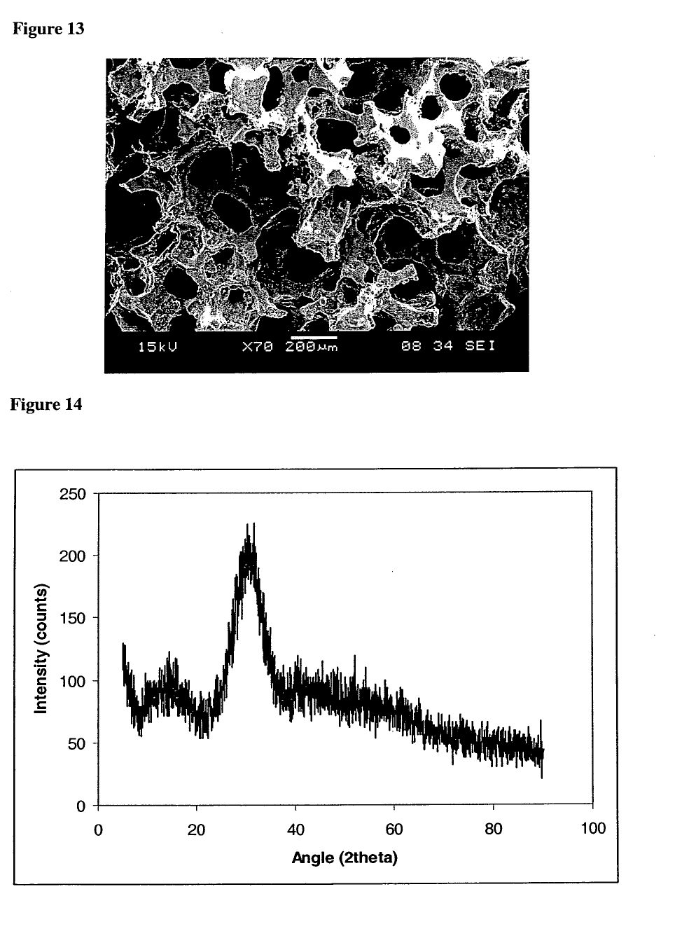

FIG. 13 shows a SEM image of an ICIE16M gel-cast foam scaffold after sintering, produced using a particle size of <38 μm.

FIG. 14 shows an XRD trace of an ICIE16M gel cast scaffold. The amorphous halo and lack of sharp peaks indicates that the material was still amorphous after sintering.

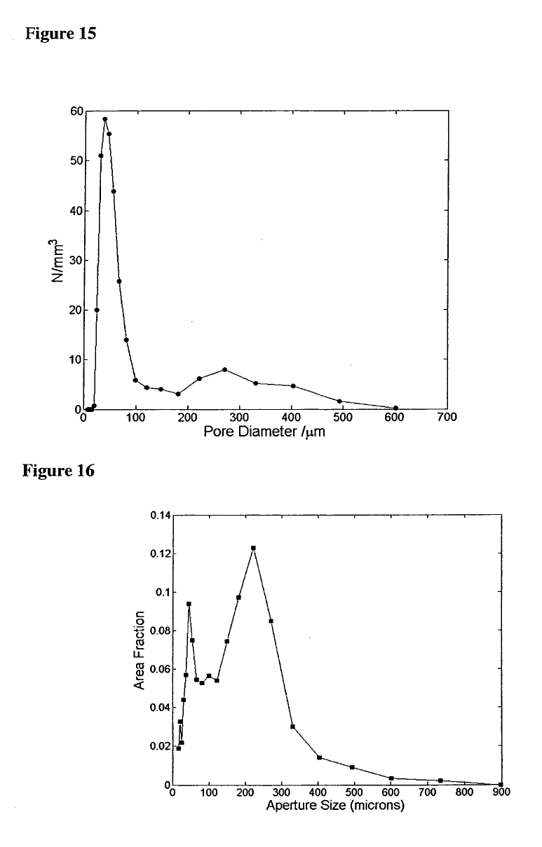

FIG. 15 shows the pore size distribution of a typical gel cast bioactive glass (ICIE16M) scaffold. Volume fraction (number of pores per mm³) as a function of pore diameter.

FIG. 16 shows the interconnect size distribution of a typical gel cast bioactive glass (ICIE16M) scaffold. Area fraction as a function of pore diameter.

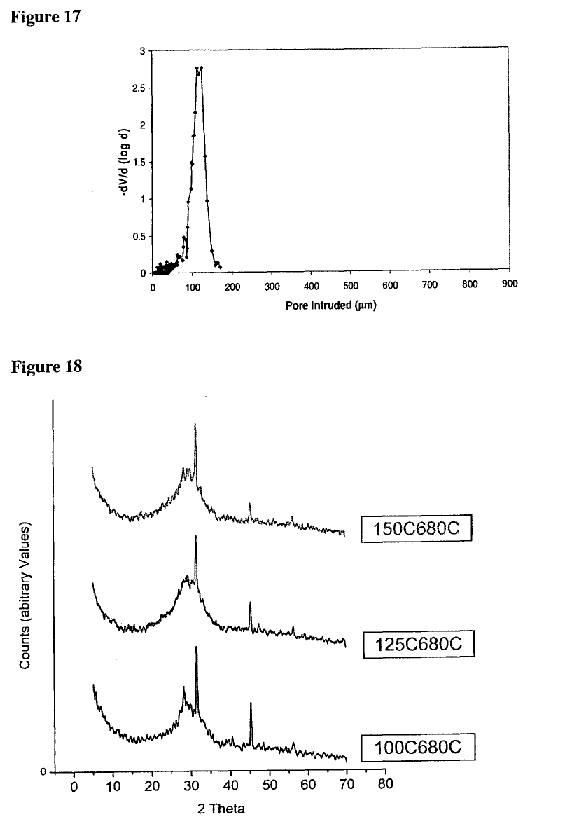

FIG. 17 shows the interconnect size distribution of a typical gel cast bioactive glass (ICIE16M) scaffold as measured by mercury porosimetry.

FIG. 18 shows XRD traces for ICIE16M scaffolds dried at different temperatures (100 C, 125 C and 150 C) and sintered at 350 C, 680 C immersed in SBF for 3 days.

The meanings of terms used herein are explained below, and the invention will now be further illustrated with reference to one or more of the following non-limiting examples.

In the context of this invention, a glass is a bioactive glass if, when implanted into living tissue, it induces formation of an interfacial bond between the glass and the surrounding tissue. An in vitro index of bioactivity is provided by the rate of development of a hydroxycarbonated apatite (HCA) layer on the surface of a glass. In certain preferred embodiments a bioactive glass is one where, on exposure of the glass to simulated body fluid (SBF), deposition of a crystalline HCA layer occurs within 3 days, more preferably within 24 hours. Deposition of a HCA layer on exposure to SBF (as described in Kokubo T., J. Biomed. Mater. Res. 1990; 24; 721-735) is a recognised test of bioactivity.

As used herein, ‘sintering’ refers to a process in which particulate matter is heated to a sintering temperature at or above which the particles adhere to each other to form a bulk solid.

As used herein, a ‘monomer’ is an organic molecule that is capable of undergoing polymerization. Monomers known in the art include acrylates, methacrylates, pyrollidones and acrylamides, for example methyl methacrylate, butyl methacrylate, acrylamide, 2-hydroxyethyl methacrylate, methyl acrylate, N-vinyl pyrollidone, ethylene dimethacrylate and diethylene glycol diacrylate. The preferred monomer for use in this invention is methyl methacrylate (MMA).

In the process of the present invention, monomer polymerisation is promoted by chemical initiation by a redox pair of an initiator, preferably a persulfate, and an amine catalyst. Known amine catalysts include N,N,N′,N′-tetramethylene diamine, N,N,N′,N′-tetra(2-hydroxyl)ethylene diamine, morpholine and 4-methyl morpholine. The preferred catalyst for use in this invention is N,N,N′,N′-tetramethylene diamine.

As used herein, a cross-linker is a compound capable of forming links with two or more polymer chains. Preferably the cross-linker is an organic molecule that forms covalent bonds with two or more polymer chains. The preferred cross-linker for use in this invention is N,N′-methylenebisacrylamide.

As used herein, the ‘gelling time’ is the time from addition of the catalyst to the time when the reaction slurry turns into a gel. The rate at which gelling occurs determines the rate of polymerisation.

### EXAMPLES

Certain glass compositions that can be utilised in the invention are set out in Table 1 below. It should, however, be appreciated that these glass compositions should not be considered limiting on the scope of the invention. The synthesis of these glass compositions and Bioglass® (for comparative purposes) is also described.

### 1. Glass Synthesis

### ICIE16M

To produce ICIE 16M, the oxides, (in mol %) 49.46% SiO₂, 27.27% CaCO₃, 6.6% Na₂CO₃, 6.6% K₂CO₃, 3% ZnO, 3% MgO, 3% SrCO₃ and 1.07% P₂O₅, were mixed together and well shaken, the mixture was then held inside a platinum crucible, which was placed in a furnace to heat up to 1400 C, and held for 1.5 hour. The mixture was then quenched in water and the coarse frit form of glass was collected and dried overnight. Other glasses were produced using a similar process, but with a firing temperature of 1370 for Bioglass® and 1420 C for ICIE 16M and a holding time of 1 hour for Bioglass® and 1.5 hours for ICIE 16M.

TABLE 1
Glass Compositions

| Compositions | 45S5 | | | ICIE | ICIE |
|---|---|---|---|---|---|
| (mol %) | (Bioglass ®) | ICIE 16 | ICIE 16M | 16 Co | 16M Co |
| SiO₂ | 46.1 | 49.46 | 49.46 | 46.48 | 46.53 |
| CaO | 26.9 | 39.27 | 27.27 | 37.58 | 27.27 |
| Na₂O | 24.4 | 6.60 | 6.60 | 6.46 | 6.47 |
| K₂O | — | 6.60 | 6.60 | 6.46 | 6.47 |
| ZnO | — | — | 3 | — | 2.94 |
| MgO | — | — | 3 | — | 2.94 |
| CoO | — | — | — | — | 1.96 |
| SrO | — | — | 3 | 1.96 | 2.94 |
| P₂O₅ | 2.6 | 1.07 | 1.07 | 1.05 | 1.05 |

300 g of original components usually produce 150 g to 200 g of glass. Calculations were made to convert the required ingredients from mol % to grams. The steps were as following:

1) Calculate how many grams of each component would weigh in 100 g of mixture, e.g. for SiO₂ in ICIE 16M it would be 49.46 mol %×60.09 (its molecular weight), which would give 29.72 g;
2) Apply the calculation to all components and sum up the results, which gave a total weight of 82.73 g in the case of ICIE 16M;
3) The required weight of mixture was 300 g, hence use 300 g over the summed up weight, 82.73 g. A ratio was obtained, which was approximately 3.626;
4) Multiply the values obtained in step 1 with the ratio, which would give the required weight of each component in order to make up a 300 g mixture.

### 2. Synthesis of Cobalt Containing Glass

To produce ICIE 16M Co, the oxides, (in mol %) 46.53% SiO₂, 27.27% CaCO₃, 6.47% Na₂CO₃, 6.47% K₂CO₃, 2.94% ZnO, 2.94% MgO, 1.96% CoCO₂, 2.94% SrCO₃ and 1.05% P₂O₅, were mixed together and well shaken, the mixture was then held inside a platinum crucible, which was placed in a furnace to heat up to 1400° C., and held for 1.5 h. The mixture was then quenched in water and the coarse frit form of glass was collected and dried overnight.

### 3. Gel Casting Variables

FIG. 2 shows a flow chart of the gel-cast foaming process that has been utilised to produce porous materials comprising the ICIE16 and ICIE16M glasses. Glass particles were mixed with water, methyl methacrylate (monomer), ammonium persulfate (APS, initiator), N,N,N′,N′-tetramethylethylene diamine (catalyst) and N,N′-methylenebisacrylamide (cross-linker). The monomer reacts with the cross-linker, in the presence of the initiator, beginning the formation of a polymer network. This increases the viscosity of the slurry and eventually binds the particles together. While the viscosity increases a surfactant is added (Triton X 100), following which a catalyst is added and the solution is foamed by vigorous agitation. The surfactant reduces the surface tension in order to stabilize the air bubbles. A foam is produced which is poured into a mould immediately prior to gelation. The material is then dried and sintered, removing the polymer binder and sintering the glass particles together, leaving a porous foam scaffold.

A key challenge was to obtain a gelling rate that allows the foaming to take place and to obtain a structure that does not collapse or crystallise on sintering. The major factors that influenced the foaming process were slurry concentration, initiator concentration, viscosity of the solution and surfactant type and quantity. The fresher the APS solution, the faster the gelling time. Sintering was affected by particle size and the thermal processing. Variation of these factors has been studied in order to optimise the gel-cast process, and arrive at the process of the invention. Results achieved by variation of these factors and consequent optimisation of these factors is discussed in the following examples. Accordingly, the process of the invention has been used to produce porous scaffolds from the ICIE16 and ICIE16M glasses.

### 3.1 Water Content Variation:

The water content of the slurry is very important. Where 14 g of glass powder was used in a total volume of 45 ml of slurry, the percentage of glass in the slurry was 31% (w/v). The foam volume is proportional to the pore sizes of the foam (ie higher foam volume equates to larger pore sizes). FIG. 3 shows that water content does not significantly affect the gelation time within a 16-22 ml range, but FIG. 4 shows that water content is critical in controlling the foam volume achieved during agitation. Using a higher concentration of powder (less water) decreased the foam volume achieved from 45 ml of slurry. Reducing the glass concentration (more water) increased the achievable foam volume, however too much water caused the construct to collapse on gelling. The volume of the slurry did not increase when 14 g of glass was agitated with 10 ml of water. When 22 ml of water was used the foam collapsed during drying. 20 ml of water was optimal (for a 45 ml total volume slurry with 14 g glass). 20 ml of water corresponds to a slurry with a glass concentration of approximately 34%. 18 ml water was optimal for a 45 ml total slurry volume with 20 g glass).

### 3.2 Glass Content Variation:

The glass content in the slurry is key to optimising the gel-cast process. Glass loadings of from 5 g to 40 g within a total slurry volume of 45 ml were utilised (representing a glass loading range from 11% to 89% w/v, where the total slurry volume is the total volume of glass, solvent, monomer, crosslinker, initiator, surfactant and catalyst prior to foaming). As the amount of glass in the system (glass loading) increases, sintering efficiency increases. However, too much glass (greater than 30 g (67% w/v)) was difficult to foam and gelling time was rapid as there was little polymer coating each particle. Too little glass (less than 10 g (22% w/v) leaves too much polymer between the glass particles which prevented sintering and caused collapse during heat treatment once the polymer was burnt out. A glass loading of 20 g (44% w/v) produced excellent results for both the glass compositions.

### 3.3 Catalyst Variation:

FIG. 5 shows that the catalyst content had a large effect on gelling time, with the system gelling faster as catalyst content increased. This had a large effect on the foam volume (FIG. 6), especially when the catalyst content increased from 5 to 6 ml, where the foam volume decreased by ˜30%. An ideal foam volume was found to be 110-130 ml.

### 3.4 Initiator Content

The APS utilised to form porous scaffolds was provided as an aqueous solution, at a concentration of 0.52 g/ml. The effect of the initiator content in the slurry on gelling time and foam volume are shown in FIGS. 7 and 8. These results were obtained for slurry 2 as shown in table 2, but with variation of the initial volume used. Increasing the APS content increases gelling time and increases foam volume. To obtain a foam volume of 110-130 ml, the APS can be provided at 1-3 ml, with particularly good results seen when provided at 2 ml.

### 3.5 Exemplary Slurry Compositions

Exemplary slurry compositions that have been used to produce a porous scaffold are, set out in Table 2. The total slurry volume was 45 ml.

TABLE 2
Summary of quantities of reagents used in the gel-
casting protocol for the ICIE16M composition:

| Order | Components | Slurry 1 | | Slurry 2 | |
|---|---|---|---|---|---|
| 1 | Glass Powder (<38 μm) | 14 | g | 20 | g |
| 2 | Ultra-purified Water | 18-20 | ml | 18 | ml |
| 3 | Methyl methacrylate | 6 | g | 6 | g |
| 4 | N,N′-methylenebisacrylamide | 3 | g | 3 | g |
| 5 | APS Solution | 4 | ml | 2 | ml |
| 6 | Dispex | 2 | drops | 2 | drops |
| 7 | Triton X100 | 0.1 | ml | 0.1 | ml |
| 8 | TEMED | 4 | ml | 4 | ml |

### 4. Heat Treatment—Drying and Sintering

The step of drying the foam acts to remove solvent from the foam prior to sintering. Where the solvent is water, residual water within the foam can cause glass particles to undergo some dissolution, releasing potassium and sodium ions from the glass. These ions can react with ammonium persulfate in the in situ polymerisation system to form sodium potassium sulfate on the glass particles. The presence of sodium potassium sulfate on and within the glass after sintering, where drying has not completely removed water, has been detected using SEM-EDX and XRD analysis.

It is desirable for the presence of the by-product sodium potassium sulfate to be avoided due to the potential of this by-product to affect bioactivity, degradation rate and mechanical properties of the glass scaffold and to alter cellular response to the scaffold material.

Increased drying temperature has been found to reduce the formation of sodium potassium sulfate due to reduced exposure of pre-sintered particles to water and therefore reduced glass dissolution. However, as drying temperatures of the constructs increased, crystallisation of the glass was observed at lower temperatures. A balance of drying and sintering will achieve optimal results.

The thermal treatment and sintering process should be optimised for the final scaffold to maintain the porous structure obtained from the gel-casting process and to achieve mechanical strength. Initially, polymer is removed by burning it out, then the glass particles must sinter together without moving out of position or crystallising. The sintering temperature (above Tg to allow viscous flow but below Tc to prevent crystallisation) was chosen from differential scanning calorimetry (DSC) traces of the glass (FIG. 9). FIG. 9 shows that as particle size decreased, the onset temperature for crystallisation decreased. Heating the foam directly to the sintering temperature, was determined to not always allow for complete burn out of the polymer. FIG. 10 shows schematics of optimised sintering procedures. Exemplary sintering procedures that have been used for the ICIE16M glass, but could be applied to other glasses, are as follows:

| Procedure A | Procedure B |
|---|---|
| T1 - 180° C. (reached at ramp of 2° C./min) | 350° C. (reached at |
| | ramp of 2° C./min) |
| t1 (first dwell time) - 1 hour | 1 hour |
| T2 - 721° C. (reached at ramp of 3° C./min) | 700° C. (reached at |
| | ramp of 3° C./min) |
| t2 (sintering time) - 0.5 hours | 1 hours |

A matrix of different drying temperatures and sintering temperature combinations were tested. Sinterability and pore network morphology were assessed by SEM and the effect of processing on devitrification was investigated with XRD.

Drying the glass at 125° C. and sintering between 680° C. and 700° C. produced particularly useful porous scaffolds with good sintering, no glass crystallisation and minimal formation of sodium potassium sulfate. These glass scaffolds were found to be bioactive, with an apatite layer forming on their surface with 3 days in SBF.

It is noteworthy that the crystallisation temperatures observed in the gel-cast system of the invention cannot be predicted merely from the theoretical values. For example, calculation of Tg and Tc onset values for ICIE16 and ICIE16M by extrapolation from DSC traces/min on glass particles at different heating rates gives the theoretical values shown in Table 3. The crystallisation onsets were extrapolated to predict the temperature the glass would begin to crystallise with zero heating rate (i.e. during a sintering hold).

TABLE 3
Summary of DSC Data:

| Parameter | ICIE16M | ICIE16 |
|---|---|---|
| Tc onset at OK min¹ (° C.) | 796 | 720 |
| Tg onset | 571 | 601 |
| Tc onset − Tg onset | 225 | 119 |
| (sintering window) | | |

Whereas ICIE16M glass particles were expected to remain amorphous until a sintering temperature of 796° C. was reached, some crystallisation was observed at lower temperatures, and the sintering temperatures used in the process of the invention are specified accordingly.

### 5. Particle Size

The particle size is critical as viscous flow sintering is driven by higher surface area. Therefore the smaller the particle size, the more readily (and closer to T_{g}) the glass will sinter. In this respect the smallest particle size possible should be used. However, crystallisation is also more likely for small particles as the glasses surface nucleate crystals, therefore the higher the surface area, the higher the risk of crystallisation. Particle size also has an effect on particle packing.

FIG. 9 shows the effect of particle size on the glass transition temperature and onset of crystallisation temperature. FIG. 12 shows a SEM of a scaffold produced using ICIE16M particles with particle sizes 140 μm sintered under the conditions shown in FIG. 10a. Outlines of particles shapes can be seen, showing that that not all the particles sintered well. However, when a particle size of 11 μm was used, the particles sintered well. FIG. 13 shows that the some of the interconnects between the macropores were in excess of 200 μm in diameter, which is suitable for vascularised bone ingrowth. A further optimised sintering protocol is shown in FIG. 10b.

FIG. 14 shows an XRD trace of an ICIE 16M gel cast scaffold. The amorphous halo and lack of sharp peaks indicates that the material was still amorphous after sintering.

Three dimensional micro computed topography (μCT) imaging of a scaffold material produced as described above demonstrates that the foaming techniques used is successful in producing a highly porous, well interconnected pore network.

### Pore Size Data

FIG. 1b shows a 3D image of a gel cast ICIE16M scaffold. Recently developed algorithms were run on the sample to obtain pore size and interconnect distributions. FIG. 15 shows the pore size distribution, for a typical gel cast glass scaffold, obtained by applying 3 algorithms (dilatation to get a distance map, watershed and top down). The distribution is bi-modal, as there are small, closed pores in the structure (<100 μm) but importantly there are many pores with diameters between 200 and 500 μm, with a mode at ˜280 μm. FIG. 16 shows the interconnect size distribution, which shows that a high percentage of interconnects are greater than 100 μm. These results imply that the scaffolds are suitable for bone regeneration.

FIG. 17 shows the interconnected pore size distribution, of a typical bioactive glass scaffold produced by the gel cast foaming process, as predicted by mercury porosimetry. Three samples were measured and the modal interconnect diameter noted. The mean of the three modal interconnect diameters was 120 μm±12 μm.

### Mechanical Properties

Glass scaffolds produced from ICIE16M using a slurry having the composition shown in Table 2 had a mean compressive strength of 2.5 MPa. This was measured with a zwick roll machine with parallel plate compression, with a load cell of 1 kN and a strain rate of 0.5 mm/min. Samples were 5 mm in diameter and 15 mm in height.

In addition, 3 sets of samples were tested: ICIE16M scaffolds dried at 100 C and 150 C, both sintered at 730 C; and ICIE16 scaffolds dried at 100 C followed by sintering at 730 C. The results were:

| | 16M 100 C. 730 C. | 16M 150 C. 730 C. | 16 100 C. 730 C. |
|---|---|---|---|
| Stress | 17 | 7 | 12 |
| (MPa) | | | |

As the drying temperature increased, the strength of the scaffold decreased, the reason is because the polymer network was disrupted when there is rapid drying taking place, and the structure after sintering is therefore more fragile. This indicates that the drying temperatures of 100-125° C. are beneficial in terms of mechanical properties. The compressive strength may be slightly higher than expected due to some crystallisation of the glasses but it is very similar that of trabecular bone strength, which is between 2-12 MPa.

### Bioactivity

Bioactivity testing was carried out on ICIE16M scaffolds made with a glass loading of 20 g in a slurry volume of 45 ml (44% w/v), i.e. slurry 2 as shown in Table 2, dried at different temperatures (100° C., 125° C. and 150° C.) and sintered at 680° C. The samples were tested in simulated body fluid (SBF), prepared according to the standard procedure as described in Kokubo T., J. Biomed. Mater. Res. 1990; 24; 721-735, at 1, 2, 4, 8, 24, 72, 168 and 336 hours. In summary, to prepare SBF the ingredients listed below were added to 750 ml of deionized water, in order, and stirred.

| Order | Chemicals | Amount/g · dm⁻³ |
|---|---|---|
| 1 | NaCl | 7.996 |
| 2 | NaHCO₃ | 0.350 |
| 3 | KCl | 0.224 |
| 4 | K₂HPO₄•3H₂O | 0.228 |
| 5 | MgCl₂•6H₂O | 0.305 |
| 6 | Diluted HCl aqueous solution | 30 ml |
| 7 | CaCl₂•2H₂O | 0.368 |
| 8 | Na₂SO₄ | 0.071 |
| 9 | (CH₂OH)₃CNH₂ | 6.057 |
| 10 | Diluted HCl aqueous solution | Heat SBF to 37° C. adjust pH |
| | | to 7.26 with HCl drops |

For the scaffolds to be considered bioactive, a hydroxycarbonate apatite (HCA) layer should form on a scaffold during immersion in SBF and preferably within one week of immersion. XRD spectra (shown in FIG. 18) for these scaffolds showed peaks identified as HCA within 3 days of immersion in SBF. Therefore, a porous scaffold produced from ICIE16M using the process of the invention creates a sintered amorphous scaffold with a suitable pore structure for a bone graft material, having good bioactivity.

It should be appreciated that various changes and modifications to the embodiments of the invention described herein will be apparent to those skilled in the art. Such changes and modifications, which can be made without departing from the spirit and the scope of the invention, fall within the scope of the invention.

1. A process for the production of a porous material, the process comprising:
a) forming a slurry comprising melt-derived glass particles, a monomer, a cross-linker and an initiator in a solvent;
b) adding a surfactant and a catalyst to the slurry;
c) agitating the slurry in the presence of a gas to generate a foam;
d) drying the foam; and
e) sintering the dried foam to provide a porous glass scaffold.

2. The process of claim 1, wherein the glass is a sinterable glass.

3. The process of claim 1, wherein the glass is a bioactive glass.

4. The process of claim 1, wherein the glass has a processing window between the glass transition temperature and the crystallisation onset temperature of at least 50° C.

5. The process of claim 1, wherein
(a) the solvent is water; and/or
(b) the monomer is methyl methacrylate (MMA); and/or
(c) the initiator is ammonium persulfate (APS) is provided as an aqueous solution; and/or
(d) the catalyst is N,N,N′,N′-tetramethylene diamine; and/or
(e) the cross-linker is N,N′-methylenebisacrylamide; and/or
(f) the surfactant is Triton X-100.

6. The process of claim 1, wherein the content of glass particles in the total volume of the slurry is from 22% to 67% (w/v).

7. The process of claim 6, wherein the content of glass particles in the total volume of the slurry is from 42 to 46% (w/v).

8. The process of claim 1 wherein the catalyst is 6.63M TEMED, provided at a 4.4% to 13.3% v/v content with respect to the total volume of the slurry.

9. The process of claim 1, wherein:
a) the monomer is present at 2.2-44.4% w/v based on the total slurry volume; and
b) the cross-linker is present at 1.1-22.2% w/v based on the total slurry volume.

10. The process of claim 1 wherein the surfactant is present at 0.0022-2.2% v/v based on the total slurry volume.

11. The process of claim 1, wherein the maximum particle size of the glass particles is no greater than 100 μm.

12. The process of claim 1, wherein the glass is formed from SiO₂ (30-60 molar %), a source of calcium (0-50 molar %), a source of sodium (0-30 molar %), a source of potassium (0-30 molar %), a source of zinc (0-10 molar %), source of magnesium (0-20 molar %) and P₂O₅ (0-14 molar %).

13. The process of claim 1, wherein the glass comprises 46-50% SiO₂ and/or wherein the glass comprises a combined molar percentage of Na₂O and K₂O which is 5-15% and/or wherein the glass comprises 20-50% CaO.

14. The process of claim 1, wherein the glass comprises a combined molar percentage of ZnO, MgO, CoO, SrO and P₂O₅ of 1-12%.

15. The process of claim 1 wherein, the glass comprises from approximately 46 to 50% SiO₂, approximately 0.5% to 1.5%, P₂O₅, approximately 0 to 2% B₂O₃, approximately 8 to 40% CaO, approximately 0 to 15% SrO, approximately 5 to 7% Na₂O, approximately 4 to 7% K₂O, approximately 0 to 4% ZnO, approximately 0-4% MgO and approximately 0 to 9% CaF₂.

16. The process of claim 1, wherein the glass comprises 2-4% ZnO and/or wherein the glass comprises 2-4% MgO.

17. The process of claim 1, wherein the glass comprises approximately 46 to 50% SiO₂, approximately 0.5% to 1.5% P₂O₅, a total molar percentage of CaO, ZnO, MgO and SrO of approximately 35-40%, approximately 5 to 7% Na₂O and approximately 5 to 7% K₂O.

18. The process of claim 1, wherein the glass comprises a source of cobalt ions at a molar percentage up to 5% and/or wherein a source of strontium ions is present, optionally wherein calcium ions are absent.

19. The process of claim 1, wherein the step of drying the foam is carried out at temperature from 50° C. to 200° C.

20. The process of claim 1, wherein the sintering process is a viscous flow sintering process.

21. The process of claim 1, wherein the sintering temperature is from 400° C. to 900° C.

22. The process of claim 1, wherein sintering is carried out in a two-step process comprising heating the foam to a first hold temperature of 80-800° C. and the increasing temperature to a sintering temperature of 400-900° C.

23. The process of claim 19, wherein the sintering process comprises heating the foam to a first hold temperature of 80-800° C. and maintaining the foam at this temperature for a first dwell time of up to 24 hours, followed by increasing the temperature to a sintering temperature of 400-900° C. and maintaining the sintering temperature for a sintering time of up to 400 hours.

24. The process of claim 1, wherein the sintering temperature is 630-730° C. and the drying temperature is 120-130° C.

25. The process of claim 21, wherein the sintering temperature is 680-700° C.

26. A porous material formed from a melt-derived glass, wherein the amorphous glass network is present within the porous material and wherein the porous material comprises macropores having a mean diameter up to 500 μm.

27. A porous material as produced by the process of claim 1.

28-29. (canceled)

30. A bone graft substitute or a tissue engineering scaffold comprising a porous material of claim 26.

31-32. (canceled)
